(19) United States
(12) Patent Application Publication
(10) Pub. No.: US 2009/0221552 A1
(43) Pub. Date: Sep. 3, 2009

(54) Methods for the Treatment of ADHD and Related Disorders

(75) Inventors: Martin Teicher, Rye, NH (US); Susan L. Andersen, Medfield, MA (US); Jacqueline A. Samson, Wellesley, MA (US)

Correspondence Address: CLARK & ELBING LLP, 101 FEDERAL STREET, BOSTON, MA, 02110, US

(73) Assignee: The McLean Hospital Corporation, Belmont, MA (US)

(21) Appl. No.: 12/224,464

(22) PCT Filed: Feb. 27, 2007
(86) PCT No.: PCT/US07/04965

§ 371 Date: May 19, 2009

Related U.S. Application Data
(60) Provisional application No. 60/777,352, filed on Feb. 28, 2006.

(51) Int. Cl. A61K 31/553; A61K 31/135; A61K 31/195; A61K 31/506; A61K 31/47; A61K 31/55; A61P 25/00

(52) U.S. Cl. 514/211.11; 514/653; 514/567; 514/256; 514/314; 514/217

(57) ABSTRACT

The invention features methods, compositions, and kits for the treatment of attention deficit hyperactivity disorder and related behavioral disorders by administering an organic cation 3 (hOCT3) inhibitor.

### BACKGROUND OF THE INVENTION

The invention relates to methods for the treatment of Attention Deficit Hyperactivity Disorder (ADHD) and other related disorders of attention or activity, including Hyperkinetic Disorder.

The noradrenergic system has been intimately associated with the modulation of higher cortical functions including attention, alertness, vigilance and executive function. Noradrenergic activation is known to profoundly affect the performance of attention, especially the maintenance of arousal, a cognitive function known to be deficient in disorders such as ADHD.

The dopamine system has been intimately associated with regulation of motor activity, reward, and selective attention. Dopaminergic activation is known to alter levels of activity and arousal, to enhance motivation, and to sharpen attention and concentration.

There are a variety of medications used for the treatment of ADHD and related disorders of attention or activity. These medications affect brain dopamine and/or norepinephrine systems. These include stimulants, e.g., methylphenidate, dextroamphetamine, cylert, and modafinil; tricyclic antidepressants, e.g., imipramine and desipramine; selective neuronal norepinephrine uptake inhibitors, e.g., atomoxetine; and/or alpha2 agonists, e.g., clonidine. All of these medications either have the potential for abuse liability; can produce undesirable side effects, e.g., tics, weight loss, sleep disturbance, cardiac effects, or blood pressure effects; and/or have a delayed onset of action.

There is a need for improved therapies for the treatment of ADHD and related behavioral disorders.

### SUMMARY OF THE INVENTION

The invention provides methods for the treatment of ADHD and related behavioral disorders. These methods include the step of blocking the organic cation transporter 3 (hOCT3) on glial cells. This approach can have several advantages over existing therapies, including the absence of addictive liability, rapid onset of efficacy, and the absence of significant adverse side effects, such as cardiovascular side effects.

The invention features a method for treating ADHD and related behavioral disorders in a subject by (a) selecting a subject having ADHD or a related behavioral disorder; and (b) administering to the subject a non-hormonal organic cation 3 (hOCT3) inhibitor in an amount sufficient to ameliorate the symptoms of said disorder. The method can further include administering a second agent within 14 days of administering the organic cation 3 (hOCT3) inhibitor, wherein the second agent is a norepinephrine uptake 1 inhibitor, direct or indirect dopamine agonist agonist, 5HT_{1A} agonist, alpha-2 agonist or antagonist, or cholinesterase inhibitor. The uptake 2 inhibitor and the second agent are administered in amounts that together are sufficient to treat said disorder.

The invention also features a composition including an organic cation 3 (hOCT3) inhibitor and second agent selected from atomoxetine, a direct or indirect dopamine agonist, a 5HT_{1A} agonist, an alpha-2 agonist or antagonist, or a cholinesterase inhibitor in amounts that together are sufficient to treat said disorder when administered to a patient.

The invention features a kit including (i) a composition comprising an organic cation 3 (hOCT3) inhibitor; and (ii) instructions for administering the composition to a patient diagnosed with ADHD or a related behavioral disorder.

The invention further features a kit including (i) an organic cation 3 (hOCT3) inhibitor; (ii) a second agent selected from a norepinephrine uptake 1 inhibitor, a direct or indirect dopamine agonist, a 5HT_{1A} agonist, an alpha-2 agonist or antagonist, or a cholinesterase inhibitor; and (iii) instructions for administering said organic cation 3 (hOCT3) inhibitor and the second agent to a patient diagnosed with ADHD or a related behavioral disorder.

Organic cation 3 (hOCT3) inhibitors that can be used in the methods, compositions, and kits of the invention include, without limitation, normetanephrine, metanephrine, 4-hydroxy-3-methoxyphenylserine (4H-3MePS), L-threo-3-(4-H-3MePS), cyanine 863, decynium-22, decynium-24, 3-O-methylisoprenaline, and prodrugs thereof. Desirably, the organic cation 3 (hOCT3) inhibitor selectively inhibits glial uptake of norepinephrine and dopamine.

The methods compositions, and kits of the invention can be useful for the treatment of ADHD and related behavioral disorders including, without limitation, Attention Deficit Hyperactivity Disorder combined, subtype, Attention Deficit Hyperactivity Disorder, predominantly hyperactive-impulsive subtype, Attention Deficit Disorder, predominantly inattentive subtype, Attention Deficit Disorder with or without hyperactivity, oppositional defiant disorder, conduct disorder and Hyperkinetic Disorder. For example, the methods, compositions, and kits of the invention can be useful for treating ADHD in patients with Tourette's Disorder, which is often comorbid with ADHD. Because the use of stimulants may worsen their tics, existing treatments for ADHD may be contraindicated in patients suffering from Tourette's Disorder.

Direct or indirect dopamine agonists that can be used in the methods, compositions, and kits of the invention include, without limitation, pergolide, bromocriptine, ropinirole, pramipexole, pemoline, cabergoline, amphetamine, dextroamphetamine and methylphenidate.

5HT_{1A} receptor agonists that can be used in the methods, compositions, and kits of the invention include, without limitation, buspirone, gepirone, ipsapirone, and flesinoxan.

Alpha-2 agonists that can be used in the methods, compositions, and kits of the invention include, without limitation, guanfacine and clonidine. Alpha-2 antagonists that can be used with the methods, compositions, and kits of the invention include, without limitation, yohimbine and idazoxan.

Norepinephrine uptake 1 inhibitors that can be used in the methods, compositions, and kits of the invention include, but are not limited to atomoxetine, reboxetine, maprotiline, bupropion, venlafaxine, amitryptiline, amoxapine, desipramine, doxepin, imipramine, nortriptyline, protriptyline, trimiprimine, and clomipramine. Desirably, the norepinephrine uptake 1 inhibitor is desipramine, imipramine, or atomoxetine.

Cholinesterase inhibitors that can be used in the methods, compositions, and kits of the invention include, without limitation, donepezil, tacrine, rivastigmine, physostigmine, galanthamine, metrifonate, neostigmine, Huperzine A, and icopezil.

The term “administration” or “administering” refers to a method of giving a dosage of a pharmaceutical composition to a human patient. The compositions of the invention can be administered by a route selected from, without limitation, inhalation, ocular, parenteral, dermal, transdermal, buccal, rectal, sublingual, perilingual, nasal, topical administration and oral administration. Parenteral administration includes intravenous, intraperitoneal, subcutaneous, and intramuscular administration. The preferred method of administration can vary depending on various factors, e.g., the components of the pharmaceutical composition and severity of the attentional or behavioral disorder.

As used herein, the term “treating” refers to administering a pharmaceutical composition for prophylactic and/or therapeutic purposes. To “prevent disease” refers to prophylactic treatment of a patient who is not yet ill, but who is susceptible to, or otherwise at risk of, ADHD or a related behavioral disorder. To “treat disease” or use for “therapeutic treatment” refers to administering treatment to a patient already suffering from ADHD or a related behavioral disorder to improve the patient's condition. Thus, in the claims and embodiments, treating is the administration to a human patient either for therapeutic or prophylactic purposes.

By “an amount sufficient” is meant the amount of a composition of the invention required to treat or prevent ADHD or a related behavioral disorder in a clinically relevant manner. A sufficient amount of an active compound used to practice the present invention for therapeutic treatment of attentional or behavioral disorders varies depending upon the manner of administration, the age, body weight, and general health of the patient. Ultimately, the prescribers will decide the appropriate amount and dosage regimen. The appropriate amounts for any therapeutic method described herein can be determined from animal models, in vitro assays, and/or clinical studies.

As used herein, “selective” refers to compounds that preferentially inhibit uptake at hOCT3 receptors (i.e., uptake 2) of norepinephrine and/or dopamine over inhibition of neuronal uptake (i.e., uptake 1) and over stimulating the release of norepinephrine, dopamine, and/or serotonin from neurons. Selectivity can be assessed in vitro, for example, by comparing the affinity and activity for any particular compound against cloned receptors engaged in glial uptake (e.g., hOCT-3 receptors) versus, neuronal uptake of norepinephrine and dopamine (hNET and hDAT), and the Potency of an agent for stimulation of release of norepinephrine, dopamine, and/or serotonin can be assessed using synpatosomes, brain slices or microdialysis in laboratory animals. Assays for hOCT3 affinity and inhibition can be performed, for example, as describe in Hayer-Zilligen et al., British Journal of Pharmacology 136:829 (2002)).

By “organic cation 3 (hOCT3) inhibitor” is meant a compound, or prodrug thereof, that blocks catecholamine uptake into the presynaptic terminal or glial cells at concentrations that are non-toxic in subjects. Examples include normetanephrine, metanephrine, 4-hydroxy-3-methoxyphenylserine (4H-3MePS), L-threo-3-(4-H-3MePS), cyanine 863, decynium-22, decynium-24, and 3-O-methylisoprenaline. Many compounds interact weakly with glial cells (e.g., neuronal norepinephrine uptake inhibitors, such as atomoxitine; stimulants, such as methylphenidate; and tricylclic antidepressants, such as desipramine and imipramine) when administered at non-toxic levels, but the interaction is too weak to alter the symptoms of ADHD and related disorders as a result of this interaction. Because norepinephrine uptake at uptake 2 sites is only inhibited by administration of such agents at toxic levels, these weakly interacting compounds are not organic cation 3 (hOCT3) inhibitors as described herein.

As used herein, “ADHD or a related behavioral disorder” refers to disorders characterized by developmentally inappropriate degrees of inattention, overactivity, and impulsivity, such as Attention Deficit Hyperactivity Disorder—combined subtype, Attention Deficit Hyperactivity Disorder—predominantly hyperactive-impulsive subtype, Attention Deficit Hyperactivity Disorder—predominantly inattentive subtype, Attention Deficit Disorder with or without hyperactivity, Hyperkinetic Disorder, oppositional defiant disorder and conduct disorder. Attention Deficit Hyperactivity Disorder is a disorder characterized by inattention, impulsiveness, and hyperactivity. This disorder can impair social function, learning and/or development and is therefore now recognized as a serious problem. It is further recognized that many children with ADHD go on to develop other comorbid conditions or social problems in adulthood. In clinical terms ADHD is diagnosed if any one of the three main clinical features, inattention, over-activity, and impulsiveness, persists in two or more situations, e.g. in both a home and school environment (American Psychiatric Association. Diagnostic and Statistical Manual of Mental Disorders, Fourth Edition (DSM-IV) Washington D.C.; American Psychiatric Association, 1994). A diagnosis of Hyperkinetic Disorder is made only if all three of the main clinical features (inattention, over-activity and impulsiveness) have been present from an early age, persist in more than one situation (e.g. home and school) and impair function (The ICD-10 Classification of Mental and Behavioural Disorders: Diagnostic Criteria for Research. Geneva: World Health Organisation, 1993: 155-7).

Other features and advantages of the invention will be apparent from the following Detailed Description and the claims.

### DETAILED DESCRIPTION

The catecholamine neurotransmitters, dopamine and norepinephrine, are released from presynaptic neurons into the synapse. The primary medications that are prescribed to treat ADHD and related behavioral disorders, such as methylphenidate, dextroamphetamine, atomoxatine, desipramine, are known to increase synaptic levels of dopamine and or norepinephrine. They do so by either inhibiting neuronal reuptake transporters for dopamine (methylphenidate) or norepinephrine (atomoxatine, desipramine), or by stimulating neuronal release of norepinephrine and dopamine (dextroamphetamine).

The invention provides a method of treating ADHD and related behavioral disorders by blocking reuptake of norepinephrine and dopamine by glial cells (“uptake 2”).

The use of uptake 2 inhibitors can be non-addictive because they do not specifically target dopamine release or neuronal reuptake of dopamine, which is the primary means of inactivating dopamine in the nucleus accumbens. Hence, these agents will not produce the rapid and dramatic rise of nucleus accumbens dopamine levels associated with addictive drugs. Furthermore, this targeting of glial uptake will result in a rapid onset of efficacy. Blocking glial uptake sites in the prefrontal cortex will exert a dramatic effect on frontal cortex dopamine, as there is an absence of neuronal dopamine transporters in prefrontal cortex. Furthermore, uptake 2 inhibitors may be combined with other agents, such as atomoxetine, to provide more rapid relief from the symptoms of ADHD and related behavioral disorders. Thus, the present methods offer several advantages over the existing therapeutic regimens for the treatment of said disorders.

Inhibitors of the Organic Cation Transporter 3 (hOCT3)

hOCT3 inhibitors include normetanephrine, cyanine 863, decynium-22, decynium-24, 3-O-methylisoprenaline, and normetananephrine precursors, such as 3 (4-hydroxy-3-methoxypheyl)-serine. These agents block the organic cation transporter 3 on glial cells, but do not affect the norepinephrine uptake 1 transporter, or the dopamine transporter, on neurons at therapeutically effective concentrations. The organic cation transporter 3 pool is very large, and blocking these glial catecholamine transporters will increase the synaptic availability of norepinephrine and dopamine throughout brain regions when norepinephrine and dopamine are released. The combined effect of increasing both norepinephrine and dopamine levels is an effective way of treating ADHD and related disorders.

hOCT3 inhibitors or precursors that are particularly useful are those that cross the blood/brain barrier where they are converted to normetanephrine, the latter being a compound that acts to inhibit hOCT3. Specific normetanephrine precursors that are useful according to the invention include, for example, those metabolized via a pathway that includes the conversion of L-threo-3-(4-hydroxy-3-methoxyphenyl)-serine (“L-threo-4H-3MePS”) into normetanephrine by an L-aromatic amino acid decarboxylase present in the brain.

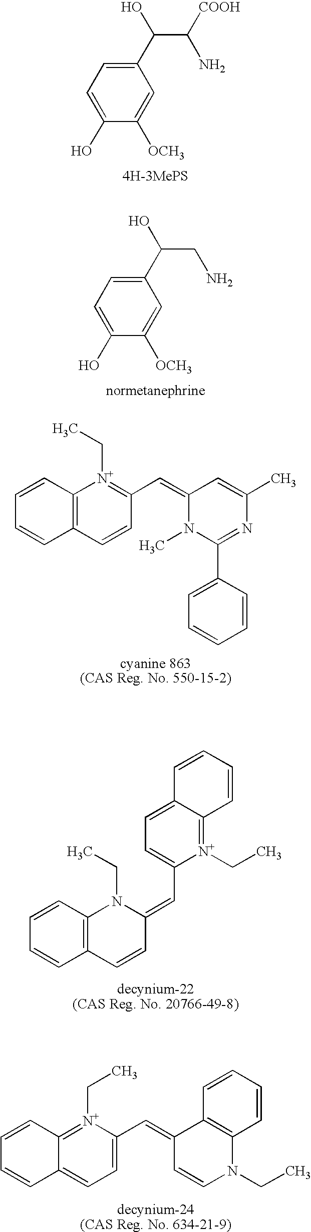

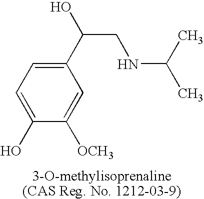

Derivatives

Derivatives of normetanephrine, decynium-22, and decynium-24 that may be used in the methods of the invention include compounds of formulas I, II, and III:

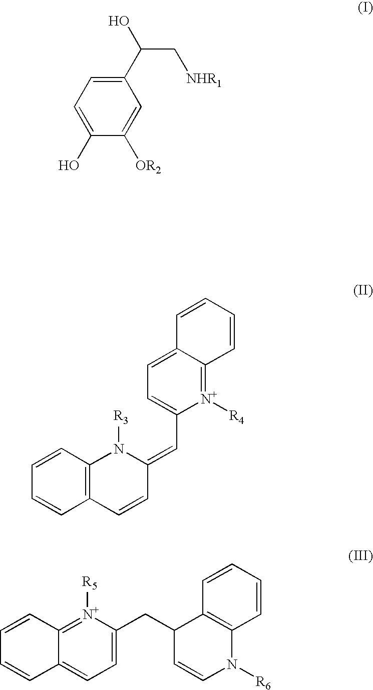

In formulas I-III, each of R₁, R₂, R₃, R₄, R₅, and R₆ is, independently, selected from methyl; ethyl; n-propyl; isopropyl; cyclopropyl; cyclopropylmethyl; cyclopropylethyl; n-butyl; iso-butyl; sec-butyl; tert-butyl; cyclobutyl; cyclobutylmethyl; cyclobutylethyl; n-pentyl; cyclopentyl; cyclopentylmethyl; cyclopentylethyl; 1-methylbutyl; 2-methylbutyl; 3-methylbutyl; 2,2-dimethylpropyl; 1-ethylpropyl; 1,1-dimethylpropyl; 1,2-dimethylpropyl; vinyl; allyl; 2-cyclopropyl-1-ethenyl; 1-propenyl; 1-butenyl; 2-butenyl; 3-butenyl; 2-methyl-1-propenyl; 2-methyl-2-propenyl; 1-pentenyl; 2-pentenyl; 3-pentenyl; 4-pentenyl; 3-methyl-1-butenyl; 3-methyl-2-butenyl; 3-methyl-3-butenyl; 2-methyl-1-butenyl; 2-methyl-2-butenyl; 2-methyl-3-butenyl; 2-ethyl-2-propenyl; 1-methyl-1-butenyl; 1-methyl-2-butenyl; 1-methyl-3-butenyl; ethynyl; 1-propynyl; 2-propynyl; 1-butynyl; 2-butynyl; 3-butynyl; 1-pentynyl; 2-pentynyl; 3-pentynyl; 4-pentynyl; 1-methyl-2-propynyl; 1-methyl-2-butynyl; 1-methyl-3-butynyl; 2-methyl-3-butynyl; 1,2-dimethyl-3-butynyl; and 2,2-dimethyl-3-butynyl.

Synthesis

The compounds identified above as useful in the methods of the invention, and their derivatives, can be prepared using methods analogous to those described in La Manna et al., Farmaco Ed. Sci. 15:9 (1960); Chapman et al., Proc. Roy. Soc. London Ser. B. 163:116 (1965); Hodgkins et al., Tetrahedron Letters 1327 (1967); Fodor et al., Acta Chim. Acad. Sci. Hung. 1:395 (1951); Axelrod et al., J. Biol. Chem. 233:697 (1958); and Heacock et al., Chem. & Ind. (London) 595 (1961).

4H-3MePS can be obtained from commercially available starting materials by the general method described in U.S. Pat. No. 3,723,514, which is incorporated herein by reference. Optically active 4H-3MePS may be obtained by reacting a derivative of racemic 4H-3MePS with an optically active base or an optically active acid, as described in the '514 patent or by using chiral chromatography.

Screening Assays

Compounds can be screened for their ability to inhibit glial uptake ((uptake 2hOCT3) using, for example, the methods described in Russ et al., Naunyn Schmiedebergs Arch. Pharmacol. 348:458 (1993) and/or Trendelenburg, U. Handb. Exp. Pharmacol. 90/I:279 (1988).

Alternatively, a compound can first be screened for the ability to bind OCT-3 using, for example, cell-free assays utilizing a form of isolated OCT-3. OCT-3 can be derived from any suitable mammalian species (e.g., human, rat, mouse, monkey). Binding of a test compound to OCT-3 protein can be determined using known methods, such as real-time Biomolecular Interaction Analysis (BIA) (Sjolander, S. and Urbaniczky, C. Anal. Chem. 63:2338 (1991); Szabo et al. Curr. Opin. Struct. Biol. 5:699 (1995)).

### Combination Therapy

Uptake 2 inhibitors can be used in combination with other agents used in the treatment of ADHD and related behavioral disorders, including norepinephrine uptake 1 inhibitors, direct or indirect dopamine agonists, 5HT_{1A} agonists, alpha-2 agonists or antagonists, and cholinesterase inhibitors to provide more rapid or greater relief from the symptoms of said disorders and to reduce the risk of adverse reactions to traditional therapies. For example, there is a delayed emergence of response to drugs that block neuronal uptake of norepinephrine because of the large pool of glial cells with hOCT3 sites. As a result, an actual increase in norepinephrine neurotransmission only occurs after the glial reuptake 2 sites become overwhelmed by the endogenous production of normetanephrine, which inhibits the organic cation 3 transporters. By directly targeting the organic cation 3 transporters, a more rapid response to the therapy is achieved. With some existing monotherapy regimens, e.g., atomoxetine, there is a subtle, gradual onset, i.e. one may not see the maximum effect of a given dose for about three weeks. This delay can be circumvented by administering both atomoxetine and an hOCT3 inhibitor as a combination therapy.

Direct or indirect dopamine agonists

Direct or indirect dopamine agonists that can be used in the methods, compositions, and kits of the invention include, without limitation, pergolide, bromocriptine, ropinirole, pramipexole, pemoline, cabergoline, amphetamine, dextroamphetamine and methylphenidate.

5HT_{1A} receptor agonists

5HT_{1A} receptor agonists that can be used in the methods, compositions, and kits of the invention include, without limitation, buspirone, gepirone, ipsapirone, and flesinoxan.

Alpha-2 agonists and antagonists

Alpha-2 agonists that can be used in the methods, compositions, and kits of the invention include, without limitation, guanfacine and clonidine. Alpha-2 antagonists that can be used with the methods, compositions, and kits of the invention include, without limitation, yohimbine and idazoxan.

Norepinephrine uptake 1 inhibitors

Norepinephrine uptake 1 inhibitors that can be used in the methods, compositions, and kits of the invention include, but are not limited to atomoxetine, reboxetine, maprotiline, bupropion, venlafaxine, amitryptiline, amoxapine, desipramine, doxepin, imipramine, nortriptyline, protriptyline, trimiprimine, and clomipramine. Desirably, the norepinephrine uptake 1 inhibitor is desipramine, imipramine, or atomoxetine.

Cholinesterase inhibitors

Cholinesterase inhibitors that can be used in the methods, compositions, and kits of the invention include, without limitation, donepezil, tacrine, rivastigmine, physostigmine, galanthamine, metrifonate, neostigmine, Huperzine A, and icopezil.

### Therapy

The invention features methods, compositions, and kits for the treatment of ADHD and related behavioral disorders.

Therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington: The Science and Practice of Pharmacy” (20th ed., ed. A. R. Gennaro, 2000, Lippincott Williams & Wilkins). Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Nanoparticulate formulations (e.g., biodegradable nanoparticles, solid lipid nanoparticles, liposomes) may be used to control the biodistribution of the compounds. Other potentially useful parenteral delivery systems include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycholate and deoxycholate, or may be oily solutions for administration in the form of nasal drops, or as a gel. The concentration of the compound in the formulation will vary depending upon a number of factors, including the dosage of the drug to be administered, and the route of administration.

The therapeutic agents described herein may be optionally administered as a pharmaceutically acceptable salt, such as a non-toxic acid addition salts or metal complexes that are commonly used in the pharmaceutical industry Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, calcium, sodium, potassium and the like.

Administration of a therapeutic agent in controlled release formulations is useful where the agent has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD50) to median effective dose (ED50)); (ii) a narrow absorption window in the gastro-intestinal tract; or (iii) a short biological half-life, so that frequent dosing during a day is required in order to sustain the plasma level at a therapeutic level.

Many strategies can be pursued to obtain controlled release of the agent. For example, controlled release can be obtained by the appropriate selection of formulation parameters and ingredients, including, e.g., appropriate controlled release compositions and coatings. Examples include single or multiple unit tablet or capsule compositions, oil solutions, suspensions, emulsions, microcapsules, microspheres, nanoparticles, patches, and liposomes.

Formulations for oral use include tablets containing the active ingredient(s) in a mixture with non-toxic pharmaceutically acceptable excipients. These excipients may be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc).

Formulations for oral use may also be provided as chewable tablets, or as hard gelatin capsules wherein the active ingredient is mixed with an inert solid diluent, or as soft gelatin capsules wherein the active ingredient is mixed with water or an oil medium.

### OTHER EMBODIMENTS

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. A method for treating ADHD or a related behavioral disorder in a subject comprising:
(a) selecting a subject having ADHD or a related behavioral disorder; and
(b) administering to the subject a non-hormonal organic cation 3 (hOCT3) inhibitor in an amount sufficient to ameliorate the symptoms of said disorder.

2. The method of claim 1, wherein said organic cation 3 (hOCT3) inhibitor is normetanephrine, metanephrine, 4-hydroxy-3-methoxyphenylserine (4H-3MePS), L-threo-3-(4-H-3MePS), cyanine 863, decynium-22, decynium-24, 3-O-methylisoprenaline, or prodrugs thereof.

3. The method of claim 1, wherein said organic cation 3 (hOCT3) inhibitor is a selective inhibitor of glial uptake of norepinephrine and/or dopamine.

4. The method of claim 1, said ADHD or related behavioral disorder is Attention Deficit Hyperactivity Disorder (combined, predominantly hyperactive-impulsive or predominantly inattentive subtypes), Attention Deficit Disorder (with or without hyperactivity), Hyperkinetic Disorder, oppositional defiant disorder, or conduct disorder.

5. The method of claim 1, further comprising administering a second agent within 14 days of administering said organic cation 3 (hOCT3) inhibitor, wherein said second agent is a norepinephrine uptake 1 inhibitor, direct or indirect dopamine agonist, 5HT_{1A} agonist, alpha-2 agonist or antagonists, or cholinesterase inhibitor, and wherein said uptake 2 inhibitor and said second agent are administered in amounts that together are sufficient to treat said disorder.

6. The method of claim 5, wherein said second agent is a norepinephrine uptake 1 inhibitor.

7. The method of claim 6, wherein said second agent is atomoxetine, reboxetine, maprotiline, bupropion, venlafaxine, amitryptiline, amoxapine, desipramine, doxepin, imipramine, nortriptyline, protriptyline, trimiprimine, or clomipramine.

8. The method of claim 7, wherein said second agent is atomoxetine.

9. The method of claim 5, wherein said second agent is a direct or indirect dopamine agonist selected from pergolide, bromocriptine, ropinirole, pramipexole, pemoline, cabergoline, amphetamine, dextroamphetamine and methylphenidate.

10. The method of claim 5, wherein said second agent is a 5HT_{1A} agonist selected from buspirone, gepirone, ipsapirone, and flesinoxan.

11. The method of claim 5, wherein said second agent is an alpha-2 agonist selected from guanfacine and clonidine or an alpha-2 antagonist selected from idazoxan and yohimbine.

12. The method of claim 5, wherein said second agent is a cholinesterase inhibitor selected from donepezil, tacrine, rivastigmine, physostigmine, galanthamine, metrifonate, neostigmine, Huperzine A, and icopezil.

13. A composition comprising an organic cation 3 (hOCT3) inhibitor and second agent selected from atomoxetine, a direct or indirect dopamine agonist, a 5HT_{1A} agonist, an alpha-2 agonist or antagonist, or a cholinesterase inhibitor in amounts that together are sufficient to treat ADHD or a related behavioral disorder when administered to a patient.

14. The composition of claim 13, wherein said organic cation 3 (hOCT3) inhibitor is normetanephrine, metanephrine, 4-hydroxy-3-methoxyphenylserine (4H-3MePS), L-threo-3-(4-H-3MePS), cyanine 863, decynium-22, decynium-24, 3-O-methylisoprenaline, or prodrugs thereof.

15. The composition of claim 13, wherein said organic cation 3 (hOCT3) inhibitor is normetanephrine.

16. The composition of claim 13, wherein said second agent is atomoxetine.

17. The composition of claim 13, wherein said second agent is a direct or indirect dopamine agonist selected from pergolide, bromocriptine, ropinirole, pramipexole, pemoline, cabergoline, amphetamine, dextroamphetamine and methylphenidate.

18. The composition of claim 13, wherein said second agent is a 5HT_{1A} agonist selected from buspirone, gepirone, ipsapirone, and flesinoxan.

19. The composition of claim 13, wherein said second agent is an alpha-2 agonist or antagonist selected from clonidine, guanfacine, yohimbine and, idazoxan.

20. The composition of claim 13, wherein said second agent is a cholinesterase inhibitor selected from donepezil, tacrine, rivastigmine, physostigmine, galanthamine, metrifonate, neostigmine, and icopezil.

21. A kit, comprising:
(i) a composition comprising an organic cation 3 (hOCT3) inhibitor; and
(ii) instructions for administering said composition to a patient diagnosed with ADHD or a related behavioral disorder.

22. A kit, comprising:
(i) an organic cation 3 (hOCT3) inhibitor;
(ii) a second agent selected from a norepinephrine uptake 1 inhibitor, a direct or indirect dopamine agonist, a 5HT_{1A} agonist, an alpha-2 agonist or antagonist, or a cholinesterase inhibitor; and
(iii) instructions for administering said organic cation 3 (hOCT3) inhibitor and said second agent to a patient diagnosed with ADHD or a related behavioral disorder.

23. The kit of claims 21 or 22, wherein said organic cation 3 (hOCT3) inhibitor is normetanephrine, metanephrine, 4-hydroxy-3-methoxyphenylserine (4H-3MePS), L-threo-3-(4-H-3MePS), cyanine 863, decynium-22, decynium-24, 3-O-methylisoprenaline, or prodrugs thereof.

24. The kit of claim 22, wherein said second agent is a norepinephrine uptake 1 inhibitor.

25. The kit of claim 22, wherein said second agent is selected from atomoxetine, reboxetine, maprotiline, bupropion, venlafaxine, amitryptiline, amoxapine, desipramine, doxepin, imipramine, nortriptyline, protriptyline, trimiprimine, and clomipramine.

26. The kit of claim 22, wherein said second agent is atomoxetine.

27. The kit of claim 22, wherein said second agent is a direct or indirect dopamine agonist selected from pergolide, bromocriptine, ropinirole, pramipexole, pemoline, cabergoline, amphetamine, dextroamphetamine and methylphenidate.

28. The kit of claim 22, wherein said second agent is a 5HT_{1A} agonist selected from buspirone, gepirone, ipsapirone, and flesinoxan.

29. The kit of claim 22, wherein said second agent is an alpha-2 agonist or antagonist selected from clonidine, guanfacine, yohimbine or, idazoxan.

30. The kit of claim 22, wherein said second agent is a cholinesterase inhibitor selected from donepezil, tacrine, rivastigmine, physostigmine, galanthamine, metrifonate, neostigmine, Huperzine A, and icopezil.
